(19) United States
(12) Patent Application Publication
(10) Pub. No.: US 2024/0057951 A1
(43) Pub. Date: Feb. 22, 2024

(54) WEIGHT BEARING COMPUTERIZED TOMOGRAPHY DEVICE

(71) Applicant: BON SECOURS MERCY HEALTH, INC., Cincinnati, OH (US)

(72) Inventor: Clayton Carmody, Toledo, OH (US)

(21) Appl. No.: 18/260,185

(22) PCT Filed: Dec. 30, 2021
(86) PCT No.: PCT/US2021/065638

§ 371 Date: Jun. 30, 2023

Related U.S. Application Data
(60) Provisional application No. 63/132,243, filed on Dec. 30, 2020.

(51) Int. Cl. A61B 6/04; A61G 13/12

(52) U.S. Cl. CPC A61B 6/0421; A61G 13/1245; A61G 13/125; A61G 13/1295; A61G 2210/50

(57) ABSTRACT

A device for simulated weight bearing on a foot and ankle of a patient, the device including a base plate configured to receive a leg of the patient. The base plate includes a base portion, a foot plate and an adjustable calf plate. The device further includes a waist belt configured to be worn by the patient and a weighting apparatus configured to selectively connect the base plate and the waist belt. The device for simulated weight bearing on a foot and ankle of a patient may be used in a method of using the device for simulated weight bearing on a foot and ankle of a patient using an imaging device.

### CROSS-REFERENCE TO RELATED APPLICATIONS

This application claims the benefit of U.S. Provisional Application Ser. No. 63/132,243, filed on Dec. 30, 2020. The entire disclosure of the above application is incorporated herein by reference.

### FIELD

The present disclosure relates generally to a device for diagnostic purposes and, more specifically, to a device for diagnostic purposes for use with imaging technologies such as computed tomography.

### INTRODUCTION

This section provides background information related to the present disclosure which is not necessarily prior art.

The foot and ankle make up a complex biomechanical system of twenty-eight bones arranged in a web of three dimensional structures, and are prone to injury and degradation from normal daily activities, as well as acute trauma and chronic conditions.

Surgeons often rely on one or more images from X-ray, computed tomography (CT), and/or magnetic resonance imaging (MRI) devices in order to diagnose foot and ankle ailments and for pre-operative planning. However, these images can be imperfect because a patient is typically lying down without weight applied to the foot and ankle structure when the images are obtained. As a result, the images do not always accurately show the foot and ankle as they may appear while bearing weight. There is increasing literature evidence to support the use of applying weight to the lower extremity while capturing these images.

It is known to obtain images of a patient in an upright, weight-bearing position in order to obtain images of the foot and ankle that properly reflect the anatomy of the foot and ankle while bearing weight. In one example, as described in U.S. Pat. No. 7,224,764 to Sukovic et. al., a CT device is configured to be in an upright position so that the patient is standing during use of the CT device. However, equipment designed for this purpose can be very cost prohibitive, often produces low quality images, and is not accessible to patients that are not able to stand upright while the images are obtained.

It is also known to simulate compression during use of a traditional CT device. For example, as described in U.S. Pat. No. 6,860,272 to Carter et. al., a device for use during an X-ray or CT scan includes an upper body plate and a lower body plate and is configured to apply pressure to the patient's skeleton. However, known devices can be cumbersome during use and difficult with respect to adjusting and customizing the devices on a patient-by-patient basis in order to optimize the images produced.

Accordingly, there is a continuing need for a device that simulates weight on a lower extremity of a patient, and that is easy to use, adjustable, customizable, and configured to be used with various imaging devices, including conventional CT devices.

### SUMMARY

In concordance with the instant disclosure, a device that simulates weight on a lower extremity of a patient, and that is easy to use, adjustable, customizable, and configured to be used with various imaging devices, including conventional CT devices, has surprisingly been discovered.

In one embodiment, a device for simulated weight bearing on a foot and ankle of a patient includes a base plate having a base portion, a foot plate, and an adjustable calf plate. The base plate is configured to receive a leg of the patient. The device further includes a waist belt configured to be worn by the patient and a weighting apparatus configured to selectively connect the base plate and the waist belt.

In another embodiment, a method of using a device for simulated weight bearing on a foot and ankle of a patient comprises a number of steps. A first step includes assembling the device. The device includes a base plate having a base portion, a foot plate, and an adjustable calf plate. The base plate is configured to receive a leg of the patient. The device further includes a waist belt configured to be worn by the patient and a weighting apparatus configured to selectively connect the base plate and the waist belt. A second step includes positioning the device on a bed of an imaging device, a third step includes positioning the patient on the device, and a fourth step includes securing the device to the patient. A fifth step includes adjusting the foot plate and the adjustable calf plate of the device to fit the patient. A sixth step includes adjusting a measure of force applied to the foot and ankle of the patient using the weighting apparatus. A seventh step includes obtaining an image of the foot and ankle of the patient, and an eighth step includes removing the device from the patient.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a top plan view of a device for simulated weight bearing on a foot and ankle of a patient according to one embodiment of the present disclosure;

FIG. 2 is a top perspective view of the device shown in FIG. 1;

FIG. 3 is a side perspective view of a device for simulated weight bearing on a foot and ankle of a patient according to another embodiment of the present disclosure, the device shown in use with a patient and an imaging device;

FIG. 4 is a side elevational view of the device shown in FIG. 1;

FIG. 5A is a top perspective view of a base plate of the device shown in FIG. 1, where a length of a base portion is defined by a position of a first base panel in relation to a second base panel;

FIG. 5B is a top perspective view of the base plate of the device shown in FIG. 5A, where an increased length of the base portion is defined by adjustment of the position of the first base panel in relation to the second base panel;

FIG. 6 is a top perspective view of a device for simulated weight bearing on a foot and ankle of a patient according to another embodiment of the present disclosure; and

FIG. 7 is a top perspective view of a base plate according to another embodiment of the present disclosure, the base plate including a jack mechanism;

FIG. 8 is a top perspective view of a base plate according to another embodiment of the present disclosure, the base plate including a ratchet mechanism;

FIG. 9 is a top perspective view of a base plate according to another embodiment of the present disclosure, the base plate including a lead screw mechanism; and

FIG. 10 is a flowchart illustrating a method of using a device for simulated weight bearing on a foot and ankle of a patient according to another embodiment of the present disclosure.

### DETAILED DESCRIPTION

The following description of technology is merely exemplary in nature of the subject matter, manufacture, and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

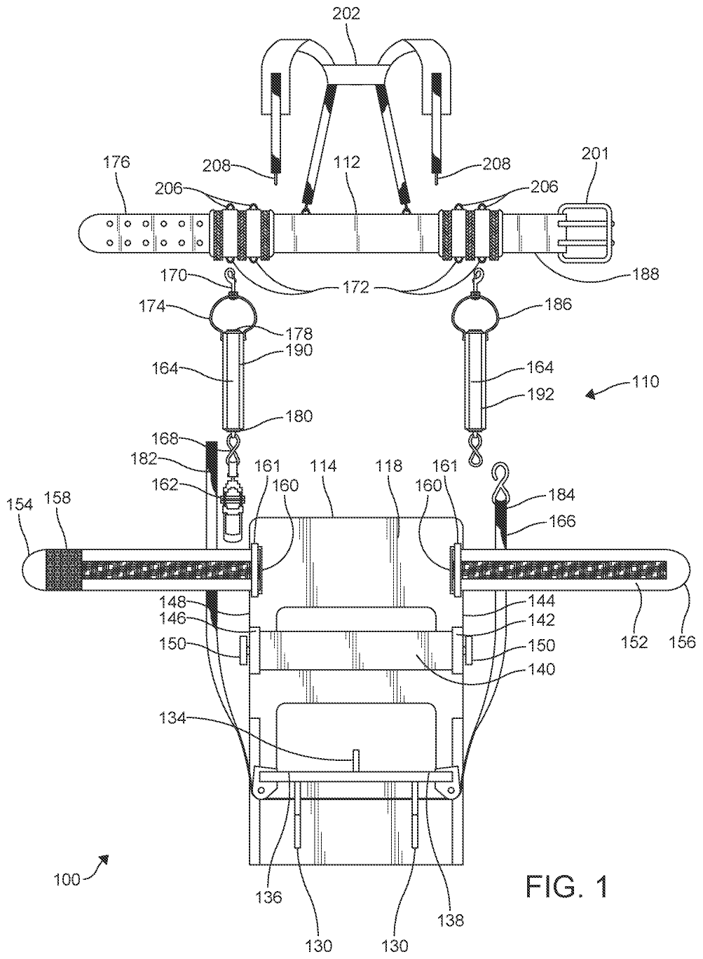

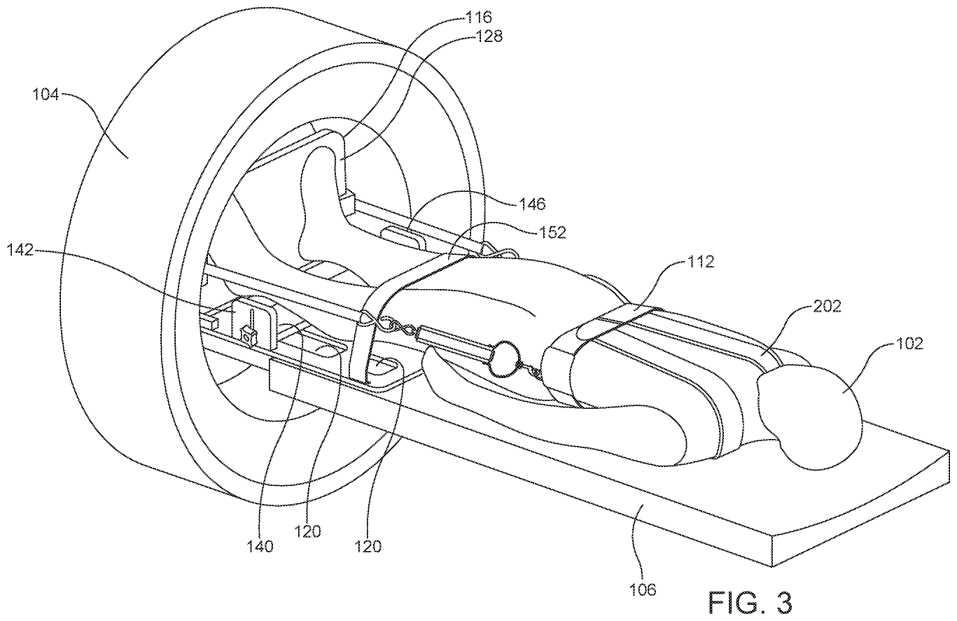

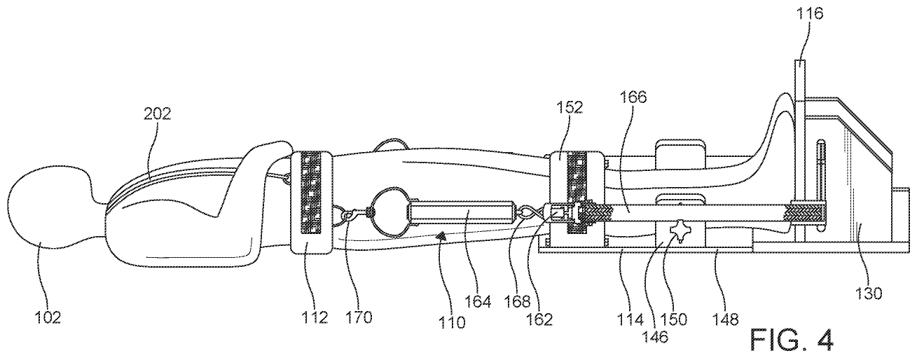

With reference to FIGS. 1-6, a device 100 for simulated weight bearing on a foot and ankle of a patient 102 is shown. The device 100 may be adapted for use with a computed tomography (CT), X-ray, magnetic resonance imaging (MRI), or other imaging device 104. More specifically, the device 100 may be configured to be positioned on a bed 106 of a CT or other imaging device 104, as shown in FIG. 3. According to certain embodiments, the device 100 may include a base plate 108, a weighting apparatus 110, and a waist belt 112.

The base plate 108 may include a base portion 114 and a foot plate 116 extending upwardly from the base portion 114. The base portion 114 and the foot plate 116 may be integral with one another or separate components that are permanently or removably affixed to one another, according to various embodiments. The base portion 114 and the foot plate 116 may be made from any rigid, lightweight material that will not interfere with the imaging device 104. In certain embodiments, the material may be non-reflective. As non-limiting examples, certain types of durable plastic, such as Delrin or Acetal, may be used.

According to various embodiments, the base portion 114 and the foot plate 116 may be made from the same material or combination of materials, or different materials, as determined by one of skill in the art. The shape, size, thickness, and configuration of the base plate 108 may be any suitable shape, size, thickness, and configuration adapted to receive and secure the foot and ankle of the patient 102. According to certain embodiments, the base plate 108 may be configured to also receive a leg, or a portion of a leg, of the patient 102.

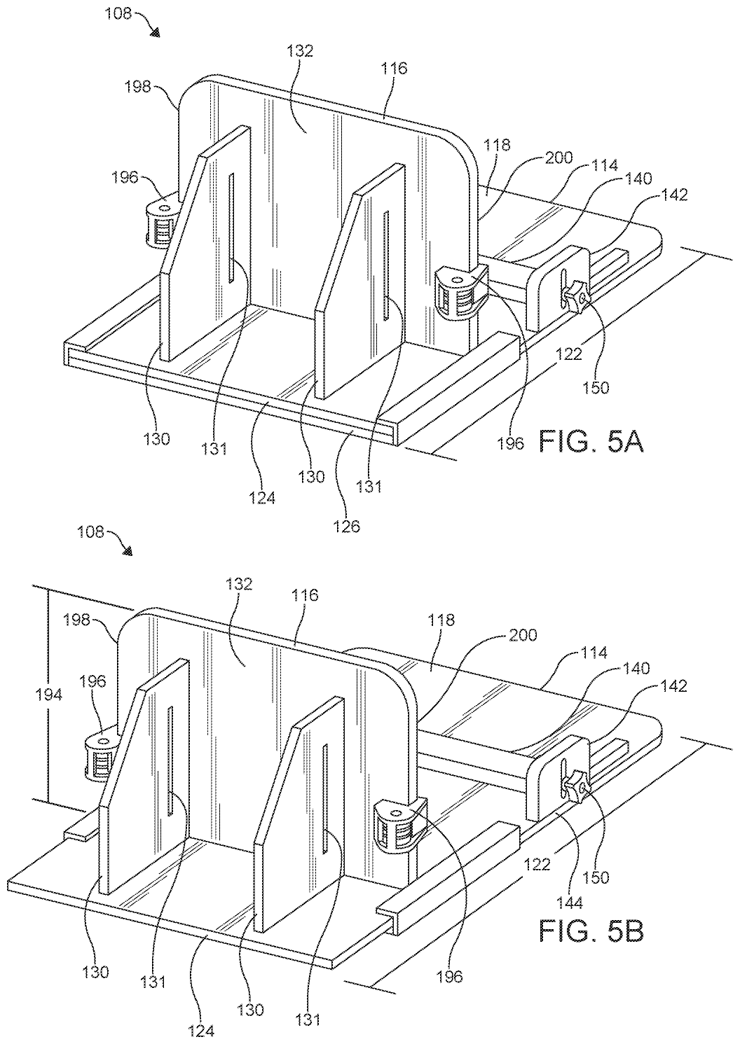

The base portion 114 of the device 100, according to certain embodiments, may have a flat configuration adapted to lay on the bed 106 of the imaging device 104. One or both of an upper surface 118 and a lower surface (not shown) of the base portion 114 may be smooth and level, or may include a textured surface, grooves, inclines, or declines, as desired. In certain embodiments, as shown in FIG. 3, the base portion 114 may have one or more apertures 120. In certain embodiments, as shown in FIGS. 5A and 5B, the base portion 114 may have a solid configuration without apertures. A length 122 of the base portion 114 may be fixed or adjustable, as shown in FIGS. 5A and 5B, as determined by one of skill in the art. As one non-limiting example, the base portion 114 may include a first base panel 124 including the foot plate 116 and a second base panel 126 capable of adjusting the length 122 of the base portion 114, as shown in FIGS. 5A and 5B. For example, the first base panel 124 can be slidably disposed relative to the second base panel 126 to adjust the length 122 of the base portion 114, where the length 122 is increased from FIG. 5A to FIG. 5B.

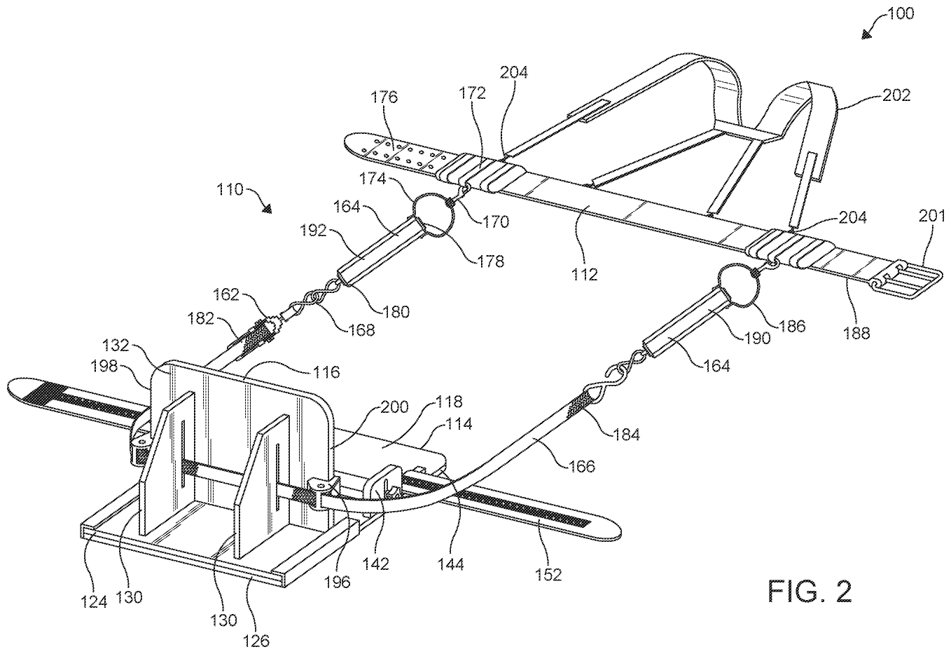

The foot plate 116 may extend outwardly from the upper surface 118 of the base portion 114, according to certain embodiments. A top surface 128 of the foot plate 116 may be adapted to receive a sole of the foot of the patient 102. As shown in FIGS. 1 and 2, the foot plate 116 may be disposed perpendicular to the base portion 114. The foot plate 116 may include a support structure 130 disposed on a bottom surface 132 of the foot plate 116. The support structure 130 may extend from the bottom surface 132 of the foot plate 116 to the upper surface 118 of the base portion 114 to provide additional stability and strength to the foot plate 116. The support structure, according to certain embodiments, may have an aperture 131.

One or both of the top surface 128 and bottom surface 132 of the foot plate 116 may be smooth and level, or may include a textured surface, grooves, inclines, or declines, as desired. In certain embodiments, the foot plate 116 may have a solid configuration without apertures, as shown in FIGS. 1-5B. In certain other embodiments, the foot plate 116 may include one or more apertures (not shown). A divider 134, as shown in FIG. 1, may be disposed centrally on the top surface 128 of the foot plate 116 and configured to divide the foot plate 116 into two sections, a first section 136 for a right foot of the patient 102 and a second section 138 for a left foot of the patient 102, according to certain embodiments.

In certain embodiments, the foot plate 116 may be adjustable. For example, the position of the foot plate 116 relative to the base portion 114 may be adjustable such that the foot of the patient 102 is able to rotate laterally, as needed, to extend or flex an ankle joint of the patient 102. The foot plate 116 may be tilted forward or backward, or may be otherwise adjustable from a position that is perpendicular to the base portion 114 to a plurality of positions that may be less than or greater than 90 degrees with respect to the base portion 114.

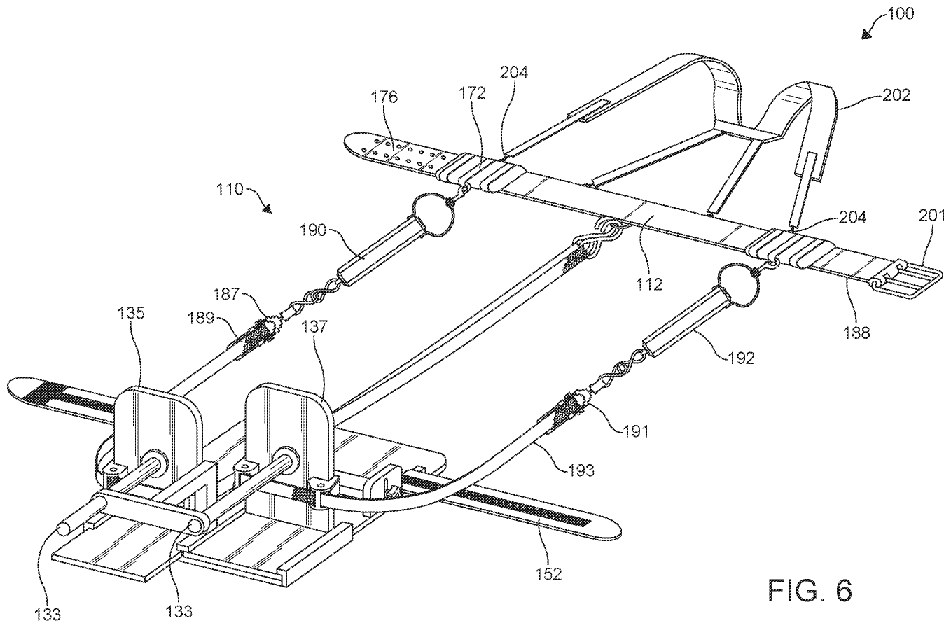

In certain embodiments, as shown in FIG. 6, the foot plate 116 may be affixed to the base portion 114 using a mounting mechanism 133. As one non-limiting example, the foot plate 116 may be affixed to the base portion 114 using a telescopic mounting mechanism 133, such that the foot plate 116 may be adjusted across multiple planes, as desired. Any suitable mounting mechanism 133 configured to allow the foot plate 116 to be adjusted, as needed, may be used to connect the foot plate 116 to the base portion 114. In certain more particular embodiments, the foot plate 116 may include a first foot plate 135 and a second foot plate 137 that may each be adjusted independently, laterally or telescopically, from one another. In certain embodiments, the mounting mechanism 133 may include a locking mechanism (not shown) to secure the foot plate 116 in a desired position with respect to the base portion 114, as desired.

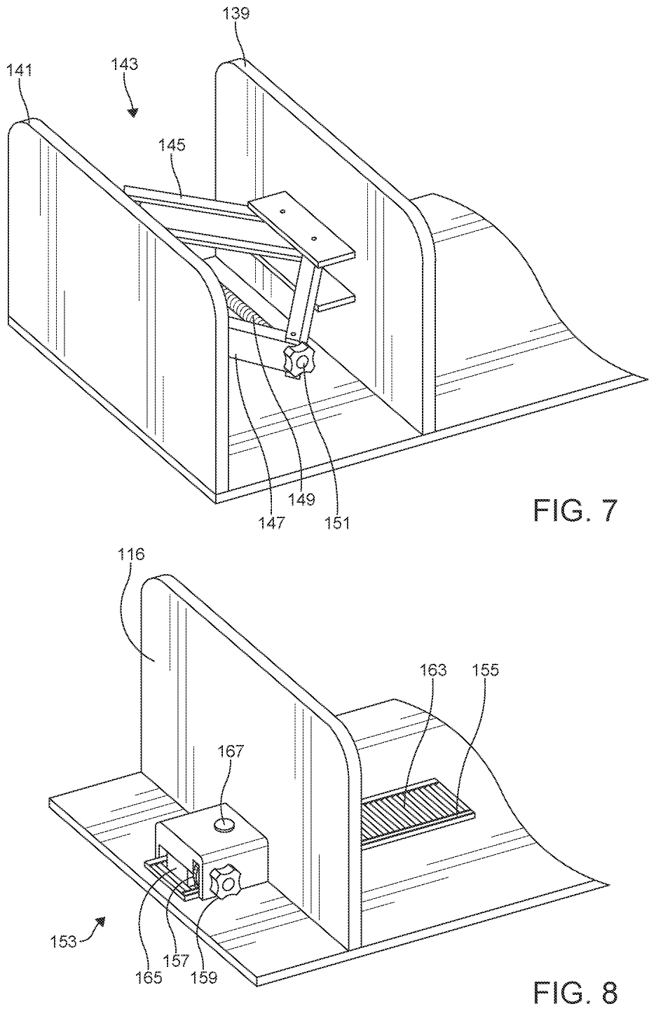

In certain embodiments, as shown in FIG. 7, a first foot plate 139 is connected to a second foot plate 141 using a jack mechanism 143. The jack mechanism 143 may include a first arm 145, a second arm 147, a threaded rod 149, and a knob 151. The jack mechanism 143 may be configured to move the first foot plate 139 closer to the second foot plate 141 in a compressed position and the first foot plate 139 away from the second foot plate 141 in an extended position using the knob 151, the first and second arms 145, 147, and the threaded rod 149. Advantageously, the first foot plate 139 is slidingly engaged with the base portion 114 and may be positioned in any desired position using the knob 151.

In certain embodiments, as shown in FIG. 8, the foot plate 116 may be slidingly connected to the base portion 114 using a ratchet mechanism 153. The ratchet mechanism 153 is configured to allow the foot plate 116 to slide along the length 122 of the base portion 114 to various positions, as desired. The ratchet mechanism 153 may include a rack 155 disposed in the base portion 114 and a pinion 157 allowing clockwise and counterclockwise movement along the rack 155 using a knob 159. According to certain embodiments, adjacent the rack 155, ratchet teeth 163 and a spring loaded pawl 165 create one way motion towards the foot of the patient 102 when the pinion 157 is turned clockwise. Advantageously, the angled ratchet teeth 163 allow the spring loaded pawl 165 to climb up the ratchet teeth 163 toward the patient 102. As the spring loaded pawl 165 moves over each peak of the ratchet teeth 163, the spring loaded pawl 165 cannot move in an opposite direction, as an interface (not shown) of the ratchet teeth 163 and spring loaded pawl 165 prevent movement in the opposite direction. A spring loaded release pin 167 is configured to lift the spring loaded pawl 165 out of the ratchet teeth 163, and the pinion 157 may then be turned counterclockwise to move the foot plate 116 away from the patient 102.

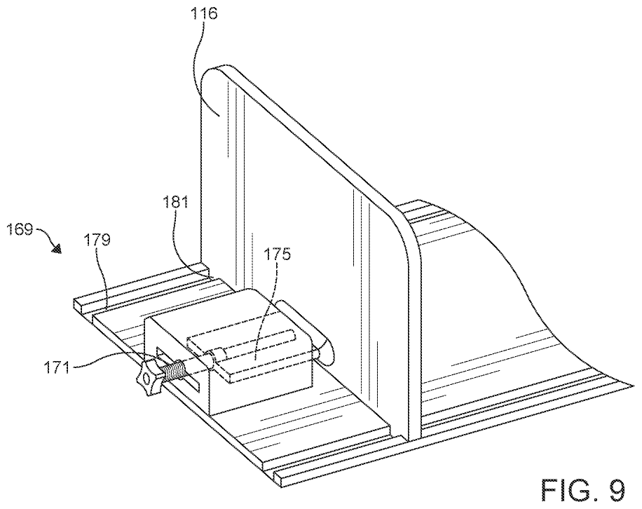

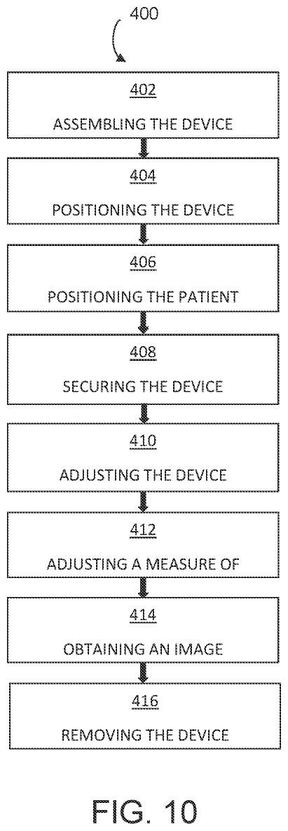

In certain embodiments, as shown in FIG. 9, the foot plate 116 may be slidingly connected to the base portion 114 using a lead screw mechanism 169. The lead screw mechanism 169 may be configured to allow the foot plate 116 to slide along the length 122 of the base portion 114 by translating rotational motion into linear motion. The lead screw mechanism 169 may include a lead screw 171 and corresponding threads (not shown) disposed in an extrusion 175 extending outwardly from the bottom surface 132 of the foot plate 116. The extrusion 175 may slidingly engage with a track (not shown) disposed in the base portion 114. As the lead screw 171 is turned clockwise, the foot plate 116 may slide along the length 122 of the base portion 114 towards the patient 102. As the lead screw 171 is turned counterclockwise, the foot plate 116 may move away from the patient 102.

In certain embodiments, a side track component 179 may be disposed on the base portion 114 and may be configured to receive a foot plate protrusion 181, thereby keeping the foot plate 116 in a preferred alignment. In certain embodiments, as needed, a self retracting strap (not shown) may be used to connect the base plate 108 to the waist belt 112, as determined by one of skill in the art.

The base plate 108 may include an adjustable calf plate 140. In certain embodiments, the calf plate 140 may be slidingly connected to a first sidewall 142 disposed on a first side 144 of the base portion 114 and a second sidewall 146 disposed on a second side 148 of the base portion 114, the first and second sides 144, 148 of the base portion 114 disposed opposite one another. Any suitable locking mechanism 150 may be used to secure the calf plate 140 to the first and second sidewalls 142, 146. As one non-limiting example, a friction fit locking mechanism 150, as shown in FIGS. 1-5B, may be used to secure the calf plate 140 in a desired position with respect to the first and second sidewalls 142, 146 of the base portion 114.

The calf plate 140 may be configured to receive a calf of the patient 102. The calf plate 140 may be adjusted upwardly away from or downwardly toward the upper surface 118 of the base portion 114 to lift or lower the calf of the patient 102, as needed, and to properly position the foot of the patient 102 with respect to the foot plate 116 and the imaging device 104. Additionally, the orientation of the calf plate 140 relative to the base portion 114 and the foot plate 116 may be adjustable, thereby providing an incline or decline, as desired. In certain embodiments (not shown), the calf plate 140 may also be adjustable along an entirety or a portion of the length 122 of the base portion 114. For example, the calf plate 140 may be slidingly engaged with the base portion 114 such that the calf plate 140 may be adjusted to fit the patient 102 and properly receive the calf of the patient 102. In certain embodiments, the first and second sidewalls 142, 146 may be slidingly engaged with the base portion 114. In certain other embodiments, the calf plate 140 may be integral with and permanently affixed to the base plate 108.

The calf plate 140 may have a smooth, flat configuration, or may include a textured surface, one or more grooves, inclines, or declines, as desired. According to various embodiments, the calf plate 140 may be fabricated using a rigid material, a flexible material, or a combination of rigid and flexible materials. In certain embodiments, the calf plate 140 may include a pad or cushion (not shown) adapted to receive the calf of the patient 102. One or more means for adjusting and locking the calf plate 140 may be included with the device 100 in order to adjust and secure the calf plate 140 in various positions.

The device 100 may further include a leg restraint belt 152. The leg restraint belt 152 may be configured to be disposed across legs of the patient 102 and to secure the legs of the patient 102 to the device 100. A first end 154 of the leg restraint belt 152 and a second end 156 of the leg restraint belt 152 may be configured to adjustably connect to one another using any suitable leg restraint belt fastener 158. As non-limiting examples, the first and second ends 154, 156 of the leg restraint belt 152 may include a hook and loop fastener or an adjustable buckle fastener. The leg restraint belt 152 may be fabricated using any flexible, durable material, such as nylon or polyester, as non-limiting examples. The leg restraint belt 152 may include a pad or cushion (not shown) in certain embodiments.

In certain embodiments, the leg restraint belt 152 may be permanently or removably connected to the base portion 114 of the base plate 108 using a belt connecting means 160, such as an aperture, as one non-limiting example. In certain embodiments, a bracket 161 may also be included. In a more particular embodiment, the base portion 114 of the base plate 108 may have belt connecting means 160 disposed on opposing sides of the base portion 114 and configured to hold the leg restraint belt 152 in place. The leg restraint belt 152 may be disposed through or affixed to the belt connecting means 160. In certain embodiments (not shown), a first leg restraint belt may be connected to a first belt connecting means disposed opposite a second leg restraint belt connected to a second belt connecting means. The first and second leg restraint belts may be configured to adjustably connect to one another.

A skilled artisan may include more than one leg restraint belt 152 with the device 100, for example, leg restraint belt 152 positioned adjacent knees of a patient and a leg restraint belt 152 positioned adjacent or affixed to the calf plate 140 of the device 100 (not shown). In certain embodiments, the leg restraint belt 152 may be adjustable along the length 122 of the base portion 114. For example, in certain embodiments (not shown), a plurality of belt connecting means 160 may be disposed along the length 122 of the base portion 114 such that the leg restraint belt 152 may be moved into different positions relative to the patient 102, as desired.

The device 100 may further include a weighting apparatus 110 configured to simulate weight on the foot and ankle of the patient 102. In certain embodiments, the weighting apparatus 110 may include a ratchet 162, a spring scale 164, and a load strap 166, for example. The ratchet 162 or any other suitable securing or locking mechanism may be removably or permanently connected to the spring scale 164 using any suitable connecting mechanism 168 and used to secure the load strap 166 in a desired position. As non-limiting examples, a clasp, buckle, or other fastener may be used as the connecting mechanism 168, as desired. The load strap 166 may be removably or slidingly connected to the ratchet 162. The load strap 166 may be fabricated from any durable, flexible material, such as nylon or polyester, as non-limiting examples.

In certain embodiments, as shown in FIG. 1, the spring scale 164 may removably connect to the waist belt 112 using a bolt or snap hook 170, or any other suitable fastening mechanism. The waist belt 112 may include a receiving mechanism 172 adapted to receive the snap hook 170 or other fastening mechanism. As non-limiting examples, the receiving mechanism 172 may be a metal ring, such as a D-ring, or a nylon loop. A skilled artisan may select any desired position for the receiving mechanism 172 on the waist belt 112, adjacent a hip of the patient as one non-limiting example, in order to more accurately distribute and simulate weight from the weighting apparatus 110. The spring scale 164 may be any measuring instrument capable of measuring force. The spring scale 164 may be mechanical or digital, according to various embodiments, and may be adapted to load up to 150 pounds on a right leg and a left leg of the patient 102. In certain embodiments, an electronic scale (not shown) may be used.

In certain embodiments, the spring scale 164 may be disposed at a first end 174 of the weighting apparatus 110. The spring scale 164 may connect to a first end 176 of the waist belt 112 at a first end 178 of the spring scale 164 and the ratchet 162 at a second end 180 of the spring scale 164. A first end 182 of the load strap 166 may connect to the ratchet 162. A second end 184 of the load strap 166 disposed at a second end 186 of the weighting apparatus 110 may connect to a second end 188 of the waist belt 112. Any suitable means for connecting the second end 184 of the load strap 166 to the second end 188 of the waist belt 112 may be employed.

In certain other embodiments, as shown in FIG. 1, the ratchet 162 and a first spring scale 190 may be connected to the first end 176 of the waist belt 112 and the first end 182 of the load strap 166. A second spring scale 192 may be connected to the second end 188 of the waist belt 112 and the second end 184 of the load strap 166. The ratchet 162, the first spring scale 190, and the second spring scale 192 may be connected to the foot plate 116 using the load strap 166. In certain embodiments, the load strap 166 may be secured to the foot plate 116 using the aperture 131. The load strap 166 may be slidingly engaged with the support structure 130 via the aperture 131 such that the load strap 166 is adjustable along a height 194 of the foot plate 116, as shown in FIG. 5B.

In certain embodiments (not shown), a first ratchet, the first spring scale 190, and a first load strap may be connected to the first side 144 of the foot plate 116. A second ratchet, the second spring scale 192, and a second load strap may be connected to the second side 148 of the foot plate 116. In certain embodiments, the first and second ratchets and the first and second spring scales 190, 192 may be connected to the foot plate 116 using a single load strap 166.

In certain other embodiments, as shown in FIG. 6, a first ratchet 187, the first spring scale 190, and a first load strap 189 may be connected to the first foot plate 135. A second ratchet 191, the second spring scale 192, and a second load strap 193 may be connected to the second foot plate 137. Advantageously, each of the first and second foot plates 135, 137 may be positioned in the same position or a different position and have the same of a different measure of force applied, as desired.

In certain embodiments, the position of the load strap 166 may be adjustable along the height 194 of the foot plate 116 such that the position of the load strap 166 may be adjusted to be level with a tibia of the patient 102. A strap guide 196 may be disposed on the foot plate 116 of the device 100 and adapted to allow the load strap 166 to slide easily and stay in a desired position and orientation relative to the device 100. In certain embodiments, the position of the strap guide 196 may be adjustable along the height 194 of the foot plate 116 on each of a first side 198 and a second side 200 of the foot plate 116. In certain embodiments, a pair of strap guides 196 may be disposed on opposing first and second sides 198, 200 of the foot plate 116. The load strap 166 may be disposed through each of the strap guides 196, such that the strap is wrapped around the foot plate 116, as shown in FIGS. 1-4. The strap guides 196 may direct the load strap 166, while holding it in position. In certain embodiments, each strap guide 196 may connect to a separate load strap 166.

The waist belt 112 may be configured to be worn around a waist of the patient 102. The first end 176 of the waist belt 112 and the second end 188 of the waist belt 112 may be configured to adjustably connect to one another. As non-limiting examples, the first and second ends 176, 188 of the waist belt 112 may include hook and loop fastener or an adjustable buckling mechanism 201. In certain embodiments, the device 100 may include suspenders 202 either permanently or removably connected to the waist belt 112. The suspenders 202 may be configured to be worn around shoulders of the patient 102 and attached to the waist belt 112 using any suitable suspender connecting means 204, as determined by one of skill in the art. The suspenders 202 may be configured to increase the stability of the device 100 on the patient 102. The waist belt 112 and the suspenders 202 may be fabricated using any flexible, durable material, such as nylon or polyester, as non-limiting examples. The waist belt 112 and the suspenders 202 may include a pad or cushion, as desired. The pad or cushion may be slidingly engaged with the waist belt 112 and the suspenders 202 thereby allowing the pad or cushion to be adjusted to fit the patient 102. In certain embodiments, the waist belt 112 may include a suspenders receiving means 206 adapted to receive a suspenders connecting means 208 disposed on the suspenders 202. As non-limiting examples, snap hooks, bolt hooks, or other suitable suspender connecting means 204 may be configured to connect with metal rings, such as b-rings, nylon loops, or other suitable suspender receiving means 206. In certain embodiments, the suspenders 202 may include a cross strap (not shown) adapted to extend from one strap to another across a collar bone of the patient 102. In certain embodiments, the suspenders 202 may connect to the first end 176 of the waist belt 112 and the second end 188 of the waist belt 112. A skilled artisan may connect the suspenders 202 to the waist belt 112 using any suitable means and configuration.

In certain embodiments, such as the embodiments shown in FIGS. 7-9, the ratchet 162 and spring scale 164 may not be necessary.

In operation, the device 100 may be used to simulate weight bearing or semi-weight bearing conditions on the foot and ankle of the patient 102 for imaging purposes. A health care provider may prepare the device 100 for use by assembling the device 100, laying the device 100 on the bed 106 of the imaging device 104, and ensuring that each of the adjustable components, such as the foot plate 116, the leg restraint belt 152, the waist belt 112, the suspenders 202, the calf plate 140, and the weighting apparatus 110 including the load strap 166, is in a position capable of receiving the patient 102 prior to any adjustments to the device 100 being made. Assembly of the device 100 may include connecting the foot plate 116 to the base portion 114, connecting the load strap 166 to the base plate 108, installing the leg restraint belt 152, connecting the base plate 108, the weighting apparatus 110, and the waist belt 112, and connecting the suspenders 202, as non-limiting examples. The modularity of the device 100 allows for easier transport, storage, and replacement of parts. For properly positioning the device 100, the health care provider may ensure that the load strap 166 is properly connected and oriented without twists or knots with respect to the base plate 108, the waist belt 112, the ratchet 162, and the spring scale 164, as one non-limiting example. In another non-limiting example, the health care provider may position the leg restraint belt 152 and the waist belt 112 in a flat, open position, as shown in FIG. 1, capable of receiving the patient 102 when the patient 102 is positioned on the device 100. In yet another non-limiting example, the calf plate 140 may be positioned directly adjacent the base portion 114 of the device 100. A health care provider may opt not to connect the suspenders 202 to the waist belt 112 when assembling the device 100 in situations where the patient 102 may have back or neck ailments or when the patient 102 requests that the suspenders 202 not be used. Likewise, a health care provider may opt to not connect the waist belt 112 and suspenders 202 to the weighting apparatus 110 if the waist belt 112 and suspenders 202 are to be fitted to the patient 102 prior to the patient 102 laying on the bed 106.

Once the device 100 is properly assembled and positioned on the bed 106 of the imaging device 104, the health care provider may help the patient 102 lay on the device 100 in preparation for adjusting the device 100 and securing the device 100 to the patient 102. Adjustments to the position of the patient 102 relative to the device 100 may be made in order to properly position the patient 102 on the device 100 prior to securing and adjusting the device 100. Proper positioning of the patient 102 may include positioning the patient 102 such that the sole of each foot of the patient is flat against the upper surface 118 of the foot plate 116 of the device 100, as one non-limiting example. As another non-limiting example, proper positioning of the patient 102 relative to the device 100 may include positioning the patient 102 so that each calf of the patient 102 is comfortably resting against the calf plate 140. Positioning the patient 102 such that the waist of the patient 102 is adjacent the waist belt 112 is yet another non-limiting example. The waist belt 112 and suspenders 202 may be secured to the patient 102 prior to the patient 102 laying on the bed 106. Advantageously, the way in which the components, such as the waist belt 112, the weighting apparatus 110, and the base plate 108, may be easily connected and disconnected to one another allows for various ways in which the patient 102 may be positioned in the device 100 during use. For example, if the waist belt 112 is secured to the patient 102 prior to the patient 102 laying on the bed 106, the waist belt 112 may be connected to the weighting apparatus 110 once the patient 102 is laying down on the bed 106. Advantageously, because of the modular, lightweight configuration of the base plate 108, the position of the base plate 108 on the bed 106 of the imaging device 104 may also be adjusted in order to properly position the device 100 and the patient 102.

The health care provider may then secure the device 100 to the patient 102 using the waist belt 112, the suspenders 202, and the leg restraint belt 152. Advantageously, the waist belt 112, the suspenders 202, and the leg restraint belt 152 are adjustable and configured to have a customizable fit specific to the patient 102.

Once the patient 102 is properly positioned in the device 100 and the device 100 is secured to the patient 102, preliminary adjustments to the device 100 may be made. Adjustments to the device 100 may include adjustments to the orientation of the foot plate 116, the placement of the foot plate 116 relative to the base portion 114 and the calf plate 140, and the position and orientation of the calf plate 140. As an example, the foot plate 116 may be adjusted across multiple planes to provide the desired angle for the images. Likewise, adjustments to the position of the foot plate 116 and the calf plate 140 relative to the base portion 114 may allow for customization of the device 100 that is specific to a height and build of the patient 102. More specifically, adjustments to the position of the calf plate 140 along the height 194 of the foot plate 116 allow for proper placement of the leg of the patient 102 with respect to the weighting apparatus 110 and the imaging device 104. Additionally, adjustments to the orientation of the load strap 166, as well as the fit of the leg restraint belt 152, the waist belt 112, and the suspenders 202 may also be made. It should be understood that the health care provider may be required to alter the position of the patient 102 relative to the device 100 and adjust various components of the device 100 any number of times and in any desired order of operation.

Once the desired adjustments to the device 100 have been made, the weighting apparatus 110 may be adjusted, as needed. Prior to applying force to the foot and leg of the patient 102 using the weighting apparatus 110, the load strap 166 may be connected to the base plate 108, the spring scale 164 may be connected to the waist belt 112, and the load strap 166 may be fed through the ratchet 162 of the weighting apparatus 110. The health care provider can connect the weighting apparatus 110 to the base plate 108 and the waist belt 112 in any order, as needed. The health care provider may eliminate a slack in the load strap 166 by pulling the load strap 166 through the ratchet 162 and subsequently advance the ratchet 162 to a desired tension of the load strap 166. When the weighting apparatus 110 is attached to the waist belt 112 and the base plate 108, the ratchet 162 may be used to increase tension in the load strap 166 by way of the spring scale 164. The tension may force the foot plate 116 against the foot of the patient 102, thereby, simulating weight bearing on the patient 102. Adjustments to the measure of force may be made by having the patient 102 bend and straighten the knee while adjusting the tension of the load strap 166 using the load strap 166 and the ratchet 162, as needed, and by advancing the ratchet 162. Adjustments to the position of the load strap 166 along the height 194 of the foot plate 116 such that a tibia of the patient 102 is level with the load strap 166 may also be made to distribute weight more accurately from the weighting apparatus 110. Likewise, adjusting the foot plate 116 and raising or lowering the calf support 140 to properly position the leg of the patient 102 relative to the device 100, the load strap 166, and the imaging device 104 may more accurately, distribute the force from the weighting apparatus 110. Advantageously, the images obtained most accurately reflect the anatomy of the foot and ankle of the patient 102 under realistic weight bearing conditions. Images may be obtained when the foot and ankle are in a desired position and subjected to a desired measure of force by the weighting apparatus 110; e.g., substantially simulating the weight of the patient 102 in a standing position.

The present technology provides certain benefits and advantages. Advantageously, the patient 102 may lie in a prone position, while still simulating weight on the foot, thereby permitting health care providers to obtain optimal images using existing imaging devices 104. Advantageously, the strap guide 196 militates against torquing and bending of the foot plate 116 caused by tension in the load strap 166 during operation. Additionally, the strap guide 196 prevents the load strap 166 for twisting and tangling, thereby ensuring proper weight distribution on the foot and leg of the patient 102. Advantageously, adjustments to the position and orientation of the calf plate 140 relative to the patient 102, the device 100, the imaging device 104, and the weighting apparatus 110, as well as the position of the load strap 166 along the height 194 of the foot plate 116 allow for proper alignment of the patient 102, the weighting apparatus 110, and the imaging device 104. Accordingly, the distribution of weight and force from the weighting apparatus 110 and positioning of the foot and leg of the patient 102 most accurately simulate a weight bearing, standing position. In certain embodiments including more than one spring scale 164, the spring scales 164 may be used jointly to ensure an even weight load on the legs of the patient 102. Alternatively, the spring scales 164 may be used to ensure a customized weight load on the legs of the patient 102. Advantageously, the device 100 may be secured to the patient 102 laying on the bed 106 of the imaging device 104 and, by simulating pressure and force on the foot and ankle of the patient 102 and thereby allowing the bones to align properly, the images obtained during the scan may be optimal for both diagnosis and surgical preparation. As a result, improved images may lead to improved outcomes.

With reference to FIG. 7, a method 400 of using the device 100, according to various embodiments, is shown. The method 400 of using the device 100 includes a first step 402 of assembling the device and a second step 404 of positioning the device 100 on the bed 106 of the imaging device 104. The method 400 further includes a third step 406 of positioning the patient 102 on the device 100. Next, the device 100 is secured to the patient 102 in a fourth step 408. In a fifth step 410, the device 100 is adjusted to form a customized fit with the patient 102. The method includes a sixth step 412 of adjusting the weighting apparatus 110 to simulate weight bearing on the foot and ankle of the patient 102 at a desired measure. Finally, a seventh step 414 includes obtaining images while the patient 102 is properly positioned in the device 100 using the imaging device 104 and an eighth step 416 includes removing the device 100 from the patient 102 once the images have been obtained.

It should be appreciated that adjustments to the device 100 may be made at any time, as needed. As one non-limiting example, adjustments to the weighting apparatus 110 or the base plate 108 may be made after obtaining the images in order to obtain additional images. It should also be appreciated that additional steps may be included in the method for using the device 100. Additional steps may include, but are not limited to, cleaning the device 100 before and after use, connecting and adjusting the waist belt 112 and suspenders 202 to the patient 102 prior to having the patient 102 lay on the bed 106, adjusting the pads, cushions, and various connecting means to properly fit the device 100 to the patient 102, connecting various components together such as clipping the spring scale 164 to the waist belt 112 or the ratchet 162, feeding the load strap 166 through the ratchet 162, creating a pre-tension on the load strap 166 by pulling any slack through the ratchet 162, observing and adjusting a measure of force displayed by the spring scale 164 to reach a desired measure of force, adjusting the measure of force using the ratchet 162, requesting that the patient 102 bend and straighten the knee while adjusting the tension of the load strap 166 using the load strap 166 and the ratchet 162, as needed, adjusting the position of the load strap 166 along the height 194 of the foot plate 116 such that the load strap 166 is level with the top of the tibia of the patient 102, adjusting the foot plate 116, and raising or lowering the calf support 140 to position the leg of the patient 102 relative to the device 100, the load strap 166, and the imaging device 104. Additional steps may also include unfastening the leg restraint belt 152 after the images have been obtained, requesting that the patient 102 bend a knee to relieve tension in the load strap 166, releasing the ratchet 162 using a release mechanism, relieving tension in the load strap 166 and spring scale 164 before releasing the ratchet 162, releasing the waist belt 112 and suspenders 202, and disconnecting various components after use. It should be further appreciated that the order of the steps included in the method and the number of times one or more of the steps may be performed is exemplary in nature and may vary. Any other suitable methods for using the device 100 may be contemplated in the present disclosure.

Example embodiments are provided so that this disclosure will be thorough, and will fully convey the scope to those who are skilled in the art. Numerous specific details are set forth such as examples of specific components, device 100s, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device 100 structures, and well-known technologies are not described in detail. Equivalent changes, modifications and variations of some embodiments, materials, compositions, and methods can be made within the scope of the present technology, with substantially similar results.

What is claimed is:

1. A device for simulated weight bearing on a foot and ankle of a patient, the device comprising:
a base plate configured to receive a leg of the patient, the base plate including a base portion, a foot plate, and an adjustable calf plate;
a waist belt configured to be worn by the patient; and
a weighting apparatus configured to selectively connect the base plate and the waist belt.

2. The device for simulated weight bearing on the foot and ankle of the patient of claim 1, wherein the foot plate is removably connected to the base portion of the base plate.

3. The device for simulated weight bearing on the foot and ankle of the patient of claim 1, wherein the foot plate is configured to be laterally adjustable to extend or flex an ankle joint of the patient.

4. The device for simulated weight bearing on the foot and ankle of the patient of claim 1, wherein a position of the foot plate is adjustable along a length of the base portion.

5. The device for simulated weight bearing on the foot and ankle of the patient of claim 4, wherein the foot plate is disposed on a first base panel of the base portion that is slidably disposed relative to a second base panel of the base portion.

6. The device for simulated weight bearing on the foot and ankle of the patient of claim 1, wherein a position of the adjustable calf plate is adjustable along a length of the base portion.

7. The device for simulated weight bearing on the foot and ankle of the patient of claim 1, wherein a position of the adjustable calf plate is adjustable along a height of the foot plate.

8. The device for simulated weight bearing on the foot and ankle of the patient of claim 1, wherein the adjustable calf plate is slidingly engaged with a sidewall disposed on a side of the base portion, and a locking mechanism is used to secure the adjustable calf plate to the sidewall in a desired position.

9. The device for simulated weight bearing on the foot and ankle of the patient of claim 1, wherein the weighting apparatus includes a load strap, a ratchet, and a spring scale.

10. The device for simulated weight bearing on the foot and ankle of the patient of claim 9, wherein the load strap is configured to connect the base plate to the weighting apparatus.

11. The device for simulated weight bearing on the foot and ankle of the patient of claim 10, wherein the load strap is threaded through an aperture disposed in the base plate.

12. The device for simulated weight bearing on the foot and ankle of the patient of claim 11, wherein a position of the load strap is adjustable relative to a height of the foot plate.

13. The device for simulated weight bearing on the foot and ankle of the patient of claim 10, wherein the load strap is connected to a strap guide disposed on the base plate.

14. The device for simulated weight bearing on the foot and ankle of the patient of claim 13, wherein a position of the strap guide is adjustable relative to a height of the foot plate.

15. The device for simulated weight bearing on the foot and ankle of the patient of claim 1, further including a pair of suspenders configured to be connected to the waist belt.

16. The device for simulated weight bearing on the foot and ankle of the patient of claim 1, wherein each of the base plate, the weighting apparatus, and the waist belt is removably connected to one another.

17. The device for simulated weight bearing on the foot and ankle of the patient of claim 1, wherein the device includes a leg restraint belt, and a position of the leg restraint belt is adjustable along a length of the base portion.

18. A method of using a device for simulated weight bearing on a foot and ankle of a patient, the method comprising steps of:
providing the device, wherein the device includes a base plate having a base portion, a foot plate, and an adjustable calf plate, the base plate configured to receive a leg of the patient, a waist belt configured to be worn by the patient, and a weighting apparatus configured to selectively connect the base plate and the waist belt;
securing the device to the patient; and
obtaining an image of the foot and ankle of the patient.

19. The method of using the device for simulated weight bearing on the foot and ankle of the patient of claim 18, wherein prior to obtaining the image of the foot and ankle of the patient, further comprising:
positioning the device on a bed of an imaging device;
positioning the patient on the device;
adjusting the foot plate and the adjustable calf plate of the device to fit the patient; and
adjusting a measure of force applied to the foot and ankle of the patient using the weighting apparatus.

20. The method of using the device for simulated weight bearing on the foot and ankle of the patient of claim 19, wherein after obtaining the image of the foot and ankle of the patient, further comprising:
performing one of:
readjusting the foot plate and the adjustable calf plate of the device to fit the patient; and
readjusting the measure of force applied to the foot and ankle of the patient using the weighting apparatus; and
obtaining another image of the foot and ankle of the patient.
